(19) United States
(12) Patent Application Publication
(10) Pub. No.: US 2003/0130231 A1
(43) Pub. Date: Jul. 10, 2003

(54) 2:1 Complex of beta- or gamma-cyclodextrin and alpha-tocopherol

(75) Inventors: Marlies Regiert, Munchen (DE); Michaela Kupka, Burghausen (DE)

Correspondence Address: COLLARD & ROE, P.C., 1077 Northern Boulevard, Roslyn, NY, 11576, US

(73) Assignee: WACKER-CHEMIE GmbH

(21) Appl. No.: 10/323,019

(22) Filed: Dec. 19, 2002

(30) Foreign Application Priority Data
Jan. 10, 2002 (DE) 102 00 657.1

(51) Int. Cl. A61K 31/724; C08B 37/16

(52) U.S. Cl. 514/58; 536/103

(57) ABSTRACT

A complex of β-cyclodextrin or γ-cyclodextrin and α-tocopherol is based upon β-cyclodextrin or γ-cyclodextrin and α-tocopherol in a molar ratio of 2:1.

### BACKGROUND OF THE INVENTION

1. Field of the Invention

The present invention relates to a 2:1 complex of β- or γ-cyclodextrin and α-tocopherol, to its preparation and use.

2. The Prior Art

Cyclodextrins are cyclic oligosaccharides made up of 6, 7 or 8 α(1-4)-linked anhydroglucose units. The α-, β- or γ-cyclodextrins prepared by enzymatic starch conversion differ in the diameter of their hydrophobic cavity and are generally suitable for the inclusion of numerous lipophilic substances.

α-Tocopherols are chroman-6-ols (3,4-dihydro-2H-1-benzopyran-6-ols) substituted in the 2-position by a 4,8,12-trimethyltridecyl radical. They occur in nature in the D-form, for example in relatively large amounts in, inter alia, wheatgerm and cottonseed oil. α-Tocopherols are slightly yellowish-reddish, oily liquids. They are insoluble in water, soluble in fats and oils, and the usual solvents for fats.

In the cosmetics industry, α-tocopherol is used as an antioxidant and active ingredient inter alia because of its moisture-binding capacity for the care of dry skin, in fat-containing cosmetic preparations, such as creams, ointments, emulsions, body and face oils, decorative cosmetics, such as lipsticks. α-Tocopherol is also effective for various skin disorders. In the food industry too, α-tocopherol is of great importance. In the human body, α-tocopherol is an important fat-soluble antioxidant which, in addition to the vitamin character, has a large number of positive physiological properties. Adults require a minimum of 4-6 mg of α-tocopherol/day in order to prevent lipid peroxidation in tissues when the supply of polyunsaturated fatty acids is moderate.

α-tocopherol is unstable toward oxidizing agents. Even in air and in the light the product darkens. This phenomenon increases in the presence of metal ions such as Fe³⁺, Ag⁺, etc. Because of this extreme sensitivity, its broad use, which is per se desired in inter alia cosmetic or dermatological products, is limited.

Stored at room temperature, α-tocopherol can be kept in the original packaging for a maximum of 12 months, and at 5° C. for up to 24 months. It is advisable to flush opened packs with inert gas and to use up the contents as quickly as possible.

Due to the sensitivity of α-tocopherol toward air and light, the more stable α-tocopherol vitamin A acid ester (α-tocopheryl acetate) is mostly used, which has lower effectiveness and thus requires higher use amounts than tocopherol. The recommended use amounts in cosmetic products are, for α-tocopherol, 0.05-0.2% by weight and, for α-tocopheryl acetate, 0.5-2596 by weight.

The effectiveness of a compound as an antioxidant is given by the radical protection factor (RPF). The measurement procedure for the radical protection factor is described by Herrling, Groth, Fuchs and Zastrow in Conference Materials “Modern Challenges To The Cosmetic Formulation” 5.5.-7-5.97, Dusseldorf, pp. 150-155, Verlag f. Chem. Ind. 1997. For α-tocopherol, the radical protection factor is 41,200, and for the customarily used α-tocopheryl acetate only 48, each×10¹⁴ radicals/mg.

JP 61014995 A2 discloses the stabilization of alpha-tocopherol using beta-cyclodextrin. The method described leads to the solubilization of the alpha-tocopherol, complexation is not described.

JP 02108622 A2 discloses a complex of γ-cyclodextrin with an α-tocopherol vitamin A acid ester which has improved solubility. However, as stated, α-tocopherol vitamin A acid does not have the same positive properties as α-tocopherol.

JP 60094403 A2 describes the preparation of a beta-cyclodextrin complex of tocopherol with diethyl ether as cosolvent. No further details are given relating to the preparation and composition of the complexes. According to the examples 1:1 complexes are formed.

The patent application EP 0835649 A1 describes a shaving composition containing a β-cyclodextrin complex of α-tocopheryl acetate.

It was the purpose of these prior art documents either to solubilize α-tocopherol or to stabilize tocopherol derivatives such as tocopheryl acetate. None of the documents mentions the stabilization of α-tocopherol toward oxygen or UV radiation. These often cause undesired discoloration of the α-tocopherol and thus also of a cosmetic product containing α-tocopherol, such as, for example, a white cream formulation.

### SUMMARY OF THE INVENTION

It is an object of the present invention to stabilize α-tocopherol against oxidative decomposition or UV-induced decomposition and thus to permit a use in pharmaceutical, dietetic and cosmetic formulations, in food technology and in the animal food sector. In particular, the discoloration of materials containing α-tocopherol, which can be attributed to the oxidation of the α-tocopherol, are also prevented.

The above object is achieved according to the present invention by providing a complex of β- or γ-cyclodextrin and α-tocopherol which comprises β- or γ-cyclodextrin and α-tocopherol in a molar ratio of 2:1.

In the complex according to the present invention, 1 mol of α-tocopherol is complexed and enclosed by 2 mol of β- or γ-cyclodextrin. It has been found entirely surprisingly and unexpected that α-tocopherol in such a complex has markedly higher stability than in a 1:1 complex of α-tocopherol and β-cyclodextrin or γ-cyclodextrin or as corresponding physical mixtures of β-cyclodextrin or γ-cyclodextrin with α-tocopherol.

The content of α-tocopherol remains largely constant in a 2:1 complex according to the present invention and in formulations comprising such a complex following storage under atmospheric oxygen and with irradiation with UVA and UVB light. The α-tocopherol is thus also available, for example in consumer products to the desired degree over prolonged periods. The complex according to the present invention thus improves the economic development and the practical handling of α-tocopherol in a desirable way.

The complex according to the present invention can be prepared, for example, in a manner known per se from a solution or using the paste method, where the weight ratio of β- or γ-cyclodextrin to α-tocopherol must be between 4:1 and 8:1, and is preferably between 5:1 and 7:1, particularly preferably in the region of 5.3:1 (for a 2:1 complex with β-cyclodextrin) or of 6.2:1 (for a 2:1 complex with γ-cyclodextrin).

The preparation of the complex from a concentrated, aqueous cyclodextrin preparation has proven advantageous. The cyclodextrin concentration of the preparation is preferably between 5 and 50% by weight. Preference is given to a cyclodextrin concentration of from 20 to 50% by weight. Depending on the consistency, the mixtures are intensively stirred or kneaded. The percent by weight of the cyclodextrin is based upon the total weight of the aqueous cyclodextrin preparation.

The reaction temperature is usually 20° C. to 80° C. Preference is given to working at 20° C. to 70° C., particularly preferably 25° C. to 65° C. The reaction time depends on the temperature and is between one hour and a few days. Preference is given to a reaction time of from 20 to 120 hours.

The complexation usually occurs under atmospheric pressure. Preferably, the complexation takes place under a protective-gas atmosphere (nitrogen or argon), and with the exclusion of daylight.

A virtually water-insoluble complex formed in this way can be used directly, although it can also be isolated and worked up by filtration, centrifugation, drying, grinding, screening, sifting, granulating and tableting.

The formulation constituents include inert and nontoxic carriers and are used for cosmetic products which comprise a complex of β- or γ-cyclodextrin with α-tocopherol in the molar ratio 2:1. These formulation constituent carriers are preferably silicone oils, such as cyclomethicones and dimethicones; humectants, i.e. substances which prevent the skin from drying out, such as propylene glycol, Mg sulfate, glycerol, emollients, i.e. substances which care for the skin, such as cetyl alcohol, liquid paraffin, petrolatum, caprylic/capric triglycerides, mineral oil, stearic acid, carbomer, beeswax, candilla wax, isopropyl and myristyl myristate, octyldodecanol, octyldodecyl lanolate, PEG-22/dodecyl glycol copolymer, hydrolyzed wheatgerm protein, gel formers, e.g.: salts of carbopol, polymethacrylates, polysaccharides, emulsifiers, e.g.: polysorbate 20, PEG-40 stearate, PEG hydrogenated castor oil, aluminum, octyl or glyceryl stearate, lecithin, preservatives, e.g.: imidazolidinylurea, chlorhexidine digluconate, phenoxyethanol, sodium benzoate, sorbic acid, methyl, ethyl, butyl parabens, BHT, BHA, sun protection filters, e.g.: 4-methylbenzylidenecamphor, DEA methoxycinnamate, benzophenone-4, octyldimethyl PABA, titanium oxide and self-tanning agents, e.g.: dihydroxyacetone, vitamins, fragrances/perfume oils, dyes, esters of mono/diglycerides of saturated fatty acids, alpha- and beta-hydroxy acids.

By “inert” it is meant that the ingredient does not react with the complex. By “nontoxic” it is meant that the ingredient is not harmful when used by the user.

The cosmetic formulations which comprise complexes of β- or γ-cyclodextrin with α-tocopherol in the molar ratio 2:1 are suitable as lotions, gels, powders, masks, creams (water-in-oil emulsions or oil-in-water emulsions), face packs, care stick, sprays, aerosols for topical applications.

The present invention thus also relates to cosmetic formulations which comprise a complex according to the present invention, along with an inert nontoxic carrier.

### BRIEF DESCRIPTION OF THE DRAWINGS

Other objects and features of the present invention will become apparent from the following detailed description considered in connection with the accompanying drawings which disclose several embodiments of the present invention. It should be understood, however, that the drawings are designed for the purpose of illustration only and not as a definition of the limits of the invention, in which:

FIG. 1 shows the solubility of cyclodextrins as a function of temperature;

FIG. 2 shows the stability of alpha-tocopherol/beta-CD complexes under UV light (UV A+B);

FIG. 3 shows the stability of alpha-tocopherol/gamma-CD complexes at 45° C.;

FIG. 4 shows the stability of alpha-tocopherol/gamma-CD complexes under UV light (UV A+B); and

FIG. 5 shows the stability of tocopherol-gamma CD complexes in cream at RT and 50° C.

### DETAILED DESCRIPTION OF PREFERRED EMBODIMENTS

The examples below serve to illustrate the invention further. The following starting materials were used:

DL-α-tocopherol is available from Merck (Darmstadt) (96-100%), molecular weight 430.7 g/mol.

Copherol® F-1300 is obtainable from Henkel (Dusseldorf) (content of DL-α-tocopherol: 86%).

CAVAMAX®) W7, β-cyclodextrin, molecular weight 1135 g/mol is obtainable from Wacker Chemie GmbH, (Munich).

CAVAMAX®) W8, γ-cyclodextrin, molecular weight 1297 g/mol is obtainable from Wacker Chemie GmbH, (Munich).

Analysis of the CD Complexes:

The guest content in the complex or a mixture of complex, freely present guest and freely present cyclodextrin can be determined quantitatively using methods such as UV, GLC, HPLC, NMR or, in the case of readily volatile oils, for a determination of the oil, as distillate.

The guest substance may be present in the reaction mixture in completely, partially or non-complexed form. If the guest is not complexed in the reaction mixture of cyclodextrin/guest, i.e. is freely available, this is referred to as a physical mixture.

The amount of complexed or freely present guest can be determined, for example, by thermogravimetry, differential scanning calorimetry (preferably for solids), drying of complexes (preferably for readily volatile substances), see Cyclodextrins in Pharmacy, Karl-Heinz Frömming, Josef Szejtli, Kluwer Academic Publishers page 87, or by eluting the freely present guest with a suitable solvent (Proceedings 7^{th} International Cyclodextrin Symposium, Tokyo, p. 207).

The latter method can likewise be used for the quantitative determination of the non-complexed, i.e. freely present, cyclodextrin. The solvent used is water.

The methods below were used to analyze the complexes/physical mixtures prepared in the examples:

Determination of the “Leachable Components”:

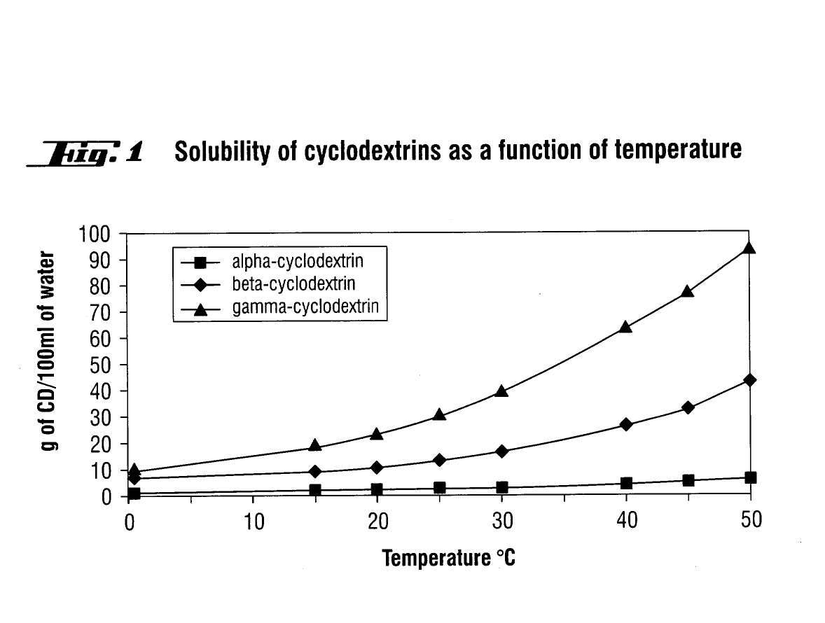

With poorly water-soluble guest substances, such as α-tocopherol, cyclodextrins form extremely sparingly soluble complexes. The large difference in solubility between these complexes and the pure cyclodextrins (FIG. 1) can be utilized in order to check whether complexation is complete. Thus, the solubility in water of β-cyclodextrin at 20° C. is 1.5%, that of γ-cyclodextrin is 18.8%, and at 25° C. 1.8% for β-cyclodextrin, and that for γ-cyclodextrin is 23%. The amount of cyclodextrin in the cyclodextrin complex which can be leached out by water corresponds to the content of free, noncomplexed cyclodextrin.

“Sun Test”

In the sun test, substances are exposed to UV A/B radiation in a targeted manner over a defined period in order to determine their resistance toward the UV A/B radiation in sunlight. Sunlight contains ultraviolet radiation of wavelength 290-320 nm (UVB) and 320-400 nm (UVA).

Functional Description of the Sun Test:

The samples are placed on a sample table. A xenon lamp is used to generate UV A and UV B radiation. The radiation is filtered through an optical filter of coated quartz shell. The part of the UV A/B radiation directed upward is reflected onto the sample by mirrors attached above the xenon lamp. If substances such as cosmetic active ingredients are subjected to the UV A/B radiation, this often leads, depending on the time, to degradation of the product even in the case of the tocopherol cited in the invention or, to a lesser extent, the α-tocopherol content of the complexes of β- or γ-cyclodextrin with α-tocopherol in accordance with the invention.

In each case 5 g of the physical mixtures, or the complexes of β- or γ-cyclodextrin with α-tocopherol prepared in the examples were inserted between two glass plates, the edges of the glass plates were sealed with sticky tape and exposed in the SUN test apparatus (ATLAS Material Testing Solutions) to the UV light (wavelength 290-320 nm UV B radiation and 320-400 nm UV A radiation) over a period of 137 hours (physical mixtures of β- or γ-cyclodextrin with α-tocopherol) or for 200 hours (complexes of β- or γ-cyclodextrin with α-tocopherol). The SUN-TEST device was fitted for this purpose with the solar standard filter ((filter in accordance with COLIPA and DIN 67501). This UV filter excludes UV C rays; thus, only UV A and B radiation reaches the sample. After the defined periods (of 0, 24, 48, 72, 137, 200 hours), samples were removed to determine the content of α-tocopherol in the complexes and physical mixtures.

### EXAMPLE 1

### Complexation of α-Tocopherol With β-CD

### a) 1:1 Beta-Cyclodextrin Complex

### Comparative Example

0.11 mol or 138 g of β-cyclodextrin (water content 10%) were mixed with 384 g of H₂O with stirring and heated to 65° C. During the heating up to 65° C., 0.11 mol or 50.6 g of α-tocopherol (content: 96.0%) were added. The mixture was stirred for 24 h at 65° C. and for about 60 h at room temperature and dried at 40° C. under vacuum or reduced pressure.

Yield: 179.45 g (95%), moisture: 5%, content of α-tocopherol according to HPLC: 25%

b) 2:1 Cyclodextrin Complex

0.11 mol of 138 g of β-cyclodextrin (water content 10%) were mixed with 250 g of H₂O with stirring and heated to 60° C. During the heating to 65° C., 0.055 mol of α-tocopherol or 27.55 g of Copherol F1000 (86% strength) were added. The mixture was stirred for 48 h at 60° C. and for about 60 h at room temperature and dried at 40° C. under vacuum or reduced pressure.

Yield: 164 g (99%), moisture: 11%, content of α-tocopherol according to HPLC: 13%

Determination of the “Leachable Components” of the Complexes:

1.8 g of the β-cyclodextrin complex with α-tocopherol according to the invention were made up to 100 g with distilled water, stirred for 2 hours at room temperature and separated over a suction filter. The filtrate was weighed and then freeze-dried. The amount of residue which remains in the flask depends on the quality of the complex. If a physical mixture was present, 100% of the cyclodextrin used dissolved in the water or remained in the filtrate. If the cyclodextrin used for the complexation was reacted completely to give a 2:1 complex, no cyclodextrin can be detected in the water or filtrate or freeze-dried filtrate, thus the solids final weight is 0. The amount of complex used and the final weight of solid were used to calculate the percentage content of the water-leachable components of the complex compound and thus the content of free β-cyclodextrin.

Leachable components 1:1 complex 4%0; (Comparative Example)

Leachable components 2:1 complex: 7%.

### EXAMPLE 2

### Complexation of α-Tocopherol With γ-Cyclodextrin

### a) 1:1 Gamma-Cyclodextrin Complex

### Comparative Example

0.1 mol or 142.52 g of γ-cyclodextrin (water content: 9%) were mixed with 255 g of H₂O with stirring and heated to 65° C. During the heating to 65° C., 0.1 mol or 45 g of α-tocopherol were added. The mixture was heated for 24 h at 65° C. and further stirred for about 60 h at room temperature and dried at 40° C. under reduced pressure.

Yield: 176 g (93%), moisture: 5%, content of α-tocopherol according to HPLC: 24%;

b) 2:1 Cyclodextrin Complex

0.21 mol or 300 g of γ-cyclodextrin (water content: 9%) were mixed with 250 g of H₂O and 0.105 mol of α-tocopherol or 52.50 g of Copherol F1300 (86% strength) with stirring and heated at 65° C. for 24 h, cooled to room temperature and dried at 40° C. under reduced pressure.

Yield: 360 g (102%), moisture: 12%, content of α-tocopherol according to HPLC: 13%;

Determination of the “Leachable Components” of the Complexes:

10 g of the γ-cyclodextrin complex with α-tocopherol were made up to 100 g with distilled water, stirred for 2 hours at room temperature and separated over a suction filter. The filtrate was weighed and then freeze-dried. The amount used and the final weight of solid were used to calculate the percentage content of the water-leachable components of the complex compound and thus the content of free γ-cyclodextrin.

Leachable components 1:1 complex 6%; (Comparative Example)

Leachable components 2:1 complex: 4%.

### EXAMPLE 3

### Preparation of Physical Mixtures of Cyclodextrin and α-Tocopherol (Comparative Example) and Determination of the “Leachable Components” of the Physical Mixtures

a) 1:1 β-Cyclodextrin/α-Tocopherol Physical Mixture

1.5 g of β-cyclodextrin and 0.5 g of α-tocopherol were homogenized in a mortar using a pestle, then made up to 100 g with water and stirred for 2 hours at RT and separated over a suction filter. The clear filtrate was weighed and then freeze-dried. The amount used and the final weight of the solid were used to calculate the percentage content of water-leachable components in the complex compound.

Leachable components: 1.45 g (73%)

HPLC was used to check the dried filtrates for free α-tocopherol, the content of α-tocopherol was <0.1%;

b) 2:1 β-Cyclodextrin/α-Tocopherol Physical Mixture

1.5 g of β-cyclodextrin and 0.3 g of Copherol F1000 (86% strength) were homogenized in a mortar using a pestle, then made up to 100 g with water and stirred for 2 hours at RT and separated over a suction filter.

The clear filtrate was weighed and then freeze-dried. The amount used and the final weight of the solid were used to calculate the percentage content of water-leachable components in the complex compound.

Leachable components: 1.4 g (77%)

HPLC was used to test the dried filtrate for free α-tocopherol, the content of α-tocopherol was <0.1%;

c) 1:1 γ-Cyclodextrin/α-Tocopherol Physical Mixture

12 g of γ-cyclodextrin and 4.5 g of α-tocopherol were homogenized in a mortar using a pestle, then made up to 100 g with water and stirred for 2 hours at RT and separated over a suction filter.

The clear filtrate was weighed and then freeze-dried. The amount used and the final weight of the solid were used to calculate the percentage content of water-leachable components in the complex compound.

Leachable components: 11 g (66.66%)

HPLC was used to test the dried filtrate for free α-tocopherol, the content of α-tocopherol was <0.1%;

d) 2:1 γ-Cyclodextrin/α-Tocopherol Physical Mixture

15 g of γ-cyclodextrin and 2.6 g of Copherol F1300 (86% strength) were homogenized in a mortar using a pestle, then made up to 100 g with water and stirred for 2 hours at RT and separated over a suction filter.

The clear filtrate was weighed and then freeze-dried. The amount used and the final weight of the solid were used to calculate the percentage content of water-leachable components in the complex compound.

Leachable components: 13.5 g (76%)

HPLC was used to test the dried filtrates for free α-tocopherol, the content of α-tocopherol was <0.1%;

### EXAMPLE 4

### Determination of the Storage Stability of α-Tocopherol as a Physical Mixture, 1:1 and 2:1 β-Cyclodextrin Complex Using the SUN Test at Room Temperature

5 g of the physical mixture or the complexes of α-tocopherol as 1:1 and 2:1 β-CD complex were introduced between two glass plates, the edges of the glass plates were sealed with sticky tape and exposed in the SUN-test device to the UV light (wavelength >290 nm) over a defined period. The content of α-tocopherol in the complexes and the physical mixtures was determined by means of HPLC.

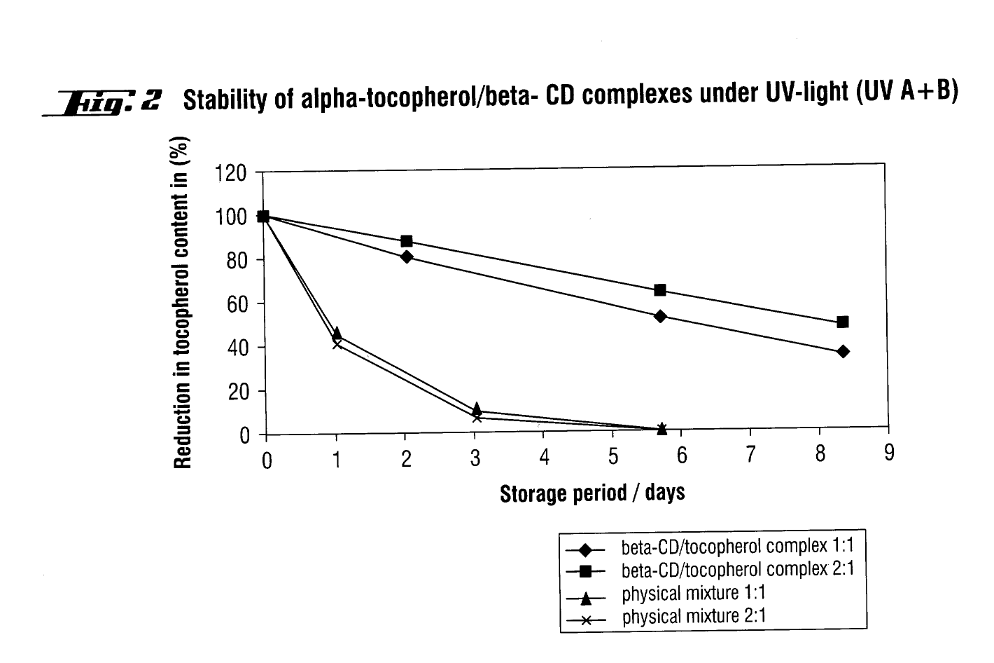

FIG. 2 shows the α-tocopherol content in the stored samples over a period of 8 months. The increased stability of the α-tocopherol in the 2:1 complex according to the invention can be clearly seen.

Color of the Complexes After 8 Months:

β-Cyclodextrin/α-tocopherol complex 1:1 slight yellowing

β-cyclodextrin/α-tocopherol complex 2:1 virtually white physical mixtures of β-cyclodextrin/α-tocopherol 2:1 intensive yellowing

physical mixtures of β-cyclodextrin/α-tocopherol 1:1 intensive yellowing

### EXAMPLE 5

### Determination of the Storage Stability of α-Tocopherol as a Physical Mixture, 1:1 and 2:1 Gamma-Cyclodextrin Complex

a) Stored at 45° C.

Complexes from Example 2 a)+b)

α-Tocopherol/γ-cyclodextrin 1:1, content of α-tocopherol: 24%

α-tocopherol/γ-cyclodextrin 2:1, content of α-tocopherol: 13%

physical mixtures of γ-cyclodextrin/α-tocopherol 1:1

physical mixtures of γ-cyclodextrin/α-tocopherol 2:1

Preparation of the Physical Mixtures

α-tocopherol/γ-cyclodextrin 1:1:0.027 mol or 38.5 g of γ-cyclodextrin (water content 9%) were weighed into a mortar and intensively ground with 0.027 mol or 11.6 g of α-tocopherol until a homogeneous powder with an α-tocopherol content of 23.15% was obtained.

α-tocopherol/γ-cyclodextrin 2:1:0.032 mol or 45.2 g of γ-cyclodextrin (water content: 9%) were weighed into a mortar and intensively ground with 0.016 mol or 6.9 g of α-tocopherol until a homogeneous powder with an α-tocopherol content of 13% was obtained.

All of the mixtures were stored in snap-on cap glassware at 45° C. in a drying cabinet. The content of α-tocopherol in the physical mixture and the complexes was determined by means of HPLC.

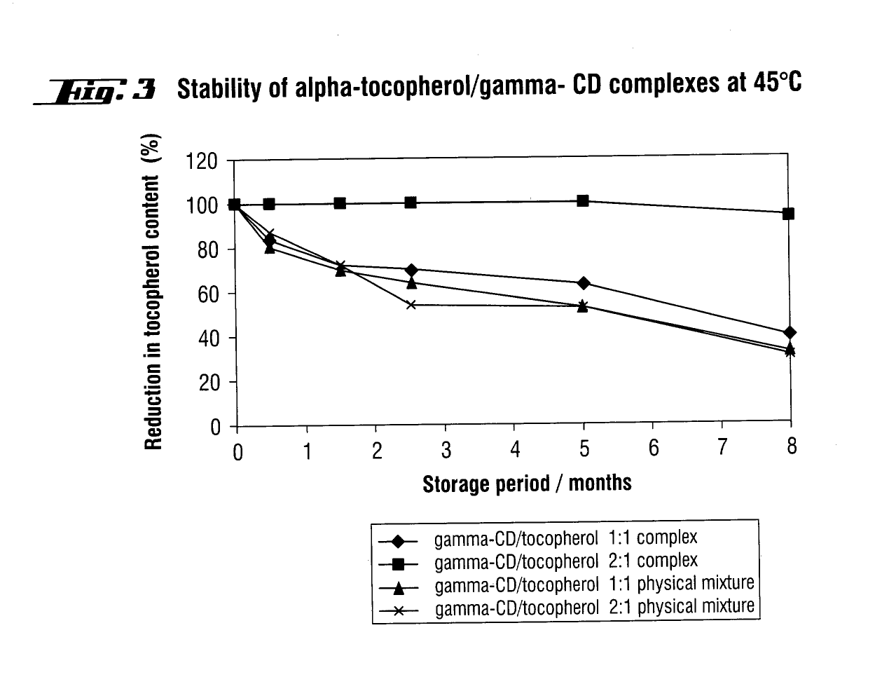

FIG. 3 shows the α-tocopherol content in the stored samples as a function of the storage period. The increased stability of the α-tocopherol in the 2:1 complex according to the invention can be clearly seen.

Color of the Complexes at the End of Storage:

γ-cyclodextrin/α-tocopherol 1:1 slight yellowing

γ-cyclodextrin/α-tocopherol 2:1 almost white

physical mixtures of γ-cyclodextrin/α-tocopherol 1:1 intensive yellow coloration

physical mixtures of γ-cyclodextrin/α-tocopherol 2:1 intensive yellow coloration

b) Stored at Room Temperature and Under UV Light (UV A+B) Wavelength >290 nm (SUN Test)

5 g of the physical mixture or of the complex of β- or γ-cyclodextrin with α-tocopherol in the molar ratio 1:1 or 2:1 were introduced between two glass plates, the edges of the glass plates were sealed with sticky tape and exposed in the SUN test device to the UV light UV A+B (wavelength>290 nm) over a defined period. After defined periods, samples were removed to determine the content of α-tocopherol in the complexes and physical mixtures.

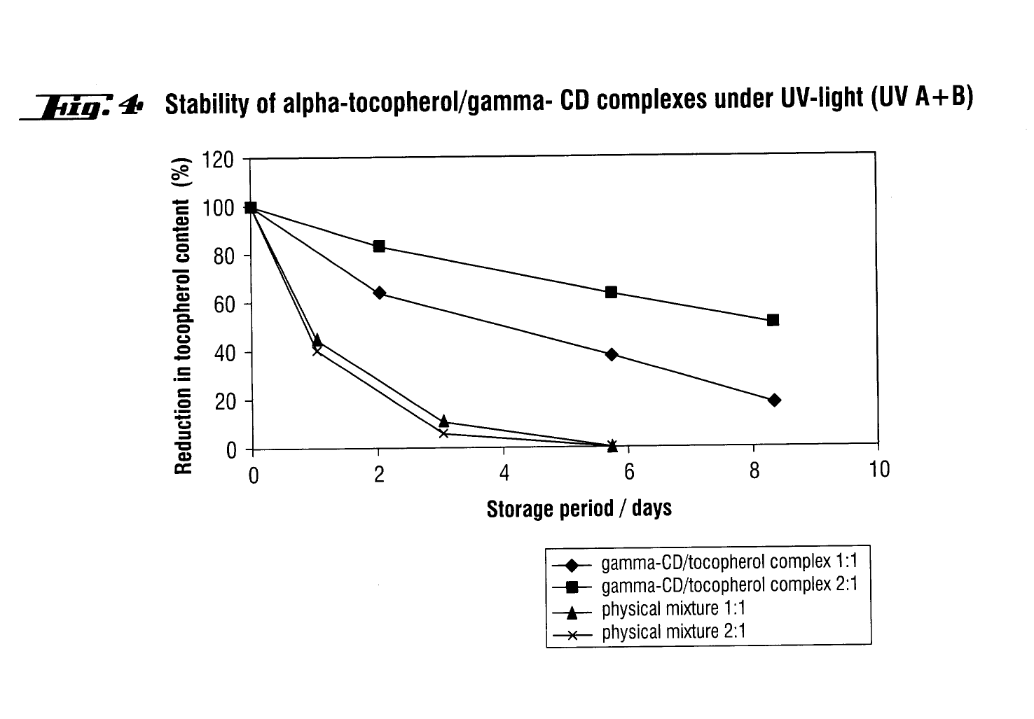

FIG. 4 shows the α-tocopherol contents in the stored samples. The increased stability of the α-tocopherol in the 2:1 complex according to the invention can be clearly seen.

Color of the Complexes at the End of Storage:

γ-cyclodextrin/α-tocopherol 1:1 slight yellowing

γ-cyclodextrin/α-tocopherol 2:1 virtually white

physical mixtures of γ-cyclodextrin/α-tocopherol 1:1 intensive yellow coloration

physical mixtures of γ-cyclodextrin/α-tocopherol 2:1 intensive yellow coloration

### EXAMPLE 6

### Storage Stability of the γ-Cyclodextrin/α-Tocopherol Complex in a Cream at RT and at 50° C.

Preparation of a Cream With 0.2% α-Tocopherol Content in a 1:1 γ-Cyclodextrin/α-Tocopherol Complex

0.4167 g of a γ-cyclodextrin/α-tocopherol complex 1:1 (24% α-tocopherol) were dispersed with 24.7251 g of water and then 24.868 g of Nivea body milk were then introduced and homogenized well. The α-tocopherol content of the cream was 0.2%. The cream was stored in cream bottles at RT and at 50° C. and the α-tocopherol content was tested over a defined period by means of HPLC.

Preparation of a Cream With a 0.2% α-Tocopherol Content in a 2:1 γ-Cyclodextrin/α-Tocopherol Complex:

0.769 g of a γ-cyclodextrin/α-tocopherol complex 2:1 (13% α-tocopherol) were dispersed with 24.579 g of water and 24.6533 g of Nivea body milk were introduced and homogenized. The content of α-tocopherol in the cream was thus adjusted to 0.2%. The cream samples were stored in cream bottles at RT and 50° C., the α-tocopherol content was tested over a defined period by means of HPLC.

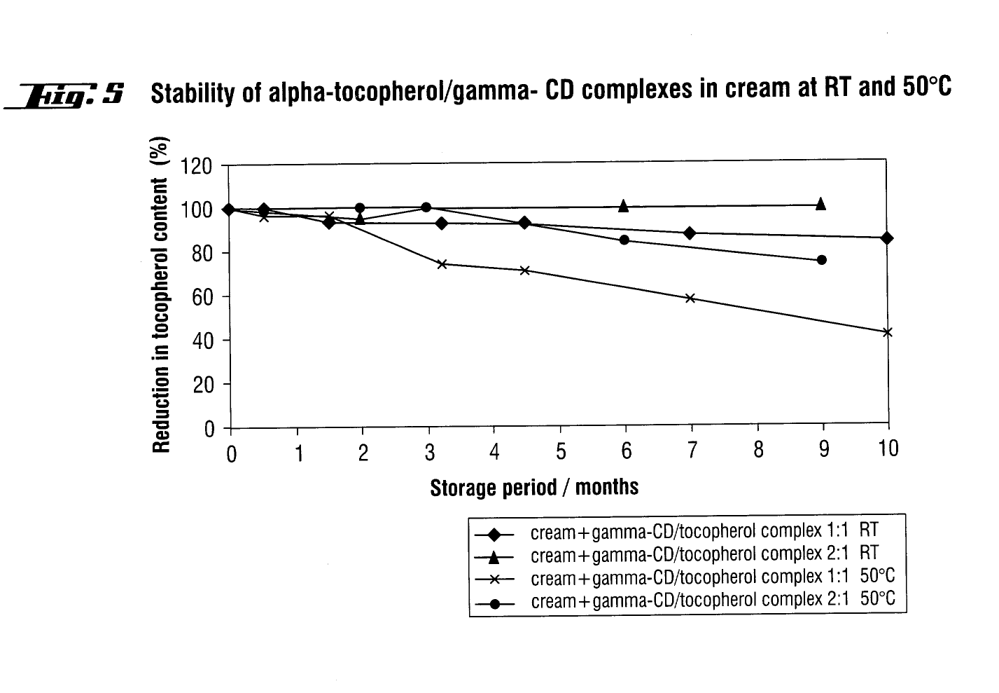

FIG. 5 shows the alpha-tocopherol contents in the stored samples.

Color of the creams containing complexes at the end of storage:

Cream containing γ-cyclodextrin/α-tocopherol 1:1 at RT slight tinting

cream containing γ-cyclodextrin/α-tocopherol 2:1 at RT white

cream containing γ-cyclodextrin/α-tocopherol 1:1 at 50° C.: slight yellow shade

cream containing γ-cyclodextrin/α-tocopherol 2:1 at 50° C.: white

### EXAMPLE 7

### Preparation of Cosmetic Formulations Containing Complexes of γ-Cyclodextrin With α-Tocopherol in the Molar Ratio 2:1

Preparation of a Sunscreen Cream With an α-Tocopherol Content of About 0.2% Using a Complex According to the Invention:

Mix components A and heat to 60° C., add B to A, homogenize well, heat D to 60° C. and mix into AB, mix in C at about 40° C.

Composition and parts by weight: and each percent by weight is based upon the total weight of the sunscreen cream.

| Active ingredients | Parts by weight |
|---|---|
| A | |
| Octyl palmitate | 2.50% |
| Octyl stearate | 3.50% |
| Polyglycerol-2 sesquiisostearate | 2.00% |
| Cyclomethicones, dimethiconol | 3.00% |
| Lauryldimethicone | 2.00% |
| Octyldimethicone ethoxyglycoside, | 12.00% |
| cyclomethicone | |
| B | |
| Titanium dioxide | 5.00% |
| Polymethylsilsesquioxane | 1.00% |
| Zinc oxide | 2.00% |
| C | |
| γ-Cyclodextrin complex 2:1, 13% | 1.54% |
| α-tocopherol (prepared in accordance | |
| with Example 2b) | |
| Glycerol | 2.00% |
| Methyl paraben | 0.10% |
| Sodium chloride | 0.40% |
| Water | 63.10% |

### EXAMPLE 8

### Preparation of an Aftersun Lotion With an α-Tocopherol Content of About 0.05% Using a Complex According to the Invention

Heat components A and B to 75° C. Incorporate A into B with stirring. After 5 min, add C. Cool to 40° C., add D and E and cool to room temperature.

Composition and parts by weight: and each percent by weight is based upon the total weight of the aftersun lotion.

| Active ingredients | Parts by weight |
|---|---|
| A | |
| Cetyl alcohol | 1.50% |
| Mineral oil | 5.00% |
| Stearic acid | 5.00% |
| B | |
| Allantoin | 0.50% |
| Propylene glycol | 3.00% |
| Water | 66.05% |
| C | |
| Cyclomethicones, dimethicones | 15.00% |
| Phenyltrimethicones | 2.00% |
| D | |
| γ-Cyclodextrin complex 2:1, 13% | 0.38% |
| α-tocopherol (prepared: | |
| Example 2b)) | |
| Water | 1.00% |
| E | |
| Phenoxyethanol, | 0.30% |
| methyl paraben, butyl paraben, | |
| ethyl paraben, propyl paraben | |
| Perfume | 0.30% |

### EXAMPLE 9

### Preparation of a Liquid Make-Up With an α-Tocopherol Content of About 0.10% Using a Complex According to the Invention

Heat A and C to 75° C., incorporate B into A using Turrax, ensure good pigment distribution, slowly emulsify C into AB using Turrax, cool to 40° C. with stirring, stir in D and E one after the other, cool to RT, homogenize cold using Turrax.

Composition and parts by weight: and each percent by weight is based upon the total weight of the liquid make-up.

| Active ingredients | Parts by weight |
|---|---|
| A | |
| White beeswax | 2.70% |
| Polyglyceryl-2 sesquiisostearate | 2.40% |
| Dimethicones | 10.00% |
| Dimethicones | 2.50% |
| Octyldimethicone ethoxyglucoside, | 11.00% |
| cyclomethicones | |
| Trimethylsiloxysilicate | 1.50% |
| B | |
| Iron oxide | 1.46% |
| Talc | 5.00% |
| Titanium dioxide; | 7.00% |
| C | |
| Sodium chloride | 2.00% |
| Water | 51.39% |
| D | |
| Methylchloroisothazolinone | 0.05% |
| Perfume | 0.30% |
| E | |
| γ-Cyclodextrin complex 2:1, 13% | 0.77% |
| α-tocopherol (prepared: | |
| Example 2b)) | |
| Water | 2.00% |

### EXAMPLE 10

### Preparation of a Body Emulsion With an α-Tocopherol Content of 0.05% by Weight

The crude materials A were introduced into a beaker, the crude materials B were mixed in a stirring vessel and heated to 65° C. The two mixtures were emulsified at 65° C. using a high-speed paddle stirrer. With further stirring, the mixture was allowed to cool to 40° C. and homogenized using an Ultra-Turrax (maximum 500 rpm). The air dissolved in the cream was removed by carefully applying a water-jet vacuum.

| Active ingredients | Parts by weight |
|---|---|
| A | |
| Glycerol monomyristate | 1.4% |
| Stearic acid | 1.2% |
| Cetyl alcohol | 0.5% |
| Isopropyl palmitate | 5% |
| γ-Cyclodextrin complex 2:1, 13% | 0.38% |
| α-tocopherol Example 2b) | |
| B | |
| Water dist. | 90.55% |
| Methyl paraben | 1% |

Each percent by weight is based upon the total weight of the body emulsion.

Accordingly, while a few embodiments of the present invention have been shown and described, it is to be understood that many changes and modifications may be made thereunto without departing from the spirit and scope of the invention as defined in the appended claims.

What is claimed is:

1. A complex selected from the group consisting of β-cyclodextrin and α-tocopherol, and γ-cyclodextrin and α-tocopherol, comprising β-cyclodextrin and α-tocopherol in a molar ratio of 2:1, or γ-cyclodextrin and α-tocopherol in a molar ratio of 2:1.

2. A process for preparing a complex selected from the group consisting of β-cyclodextrin and α-tocopherol, and γ-cyclodextrin and α-tocopherol comprising
mixing β-cyclodextrin and α-tocopherol in a weight ratio between 4:1 and 8:1; or
mixing γ-cyclodextrin and α-tocopherol in a weight ratio between 4-1 and 8:1.

3. The process as claimed in claim 2, wherein β-cyclodextrin or γ-cyclodextrin is used in the form of a concentrated, aqueous cyclodextrin preparation,
wherein the cyclodextrin concentration of the preparation is between 5 and 50% by weight based upon the total weight of the aqueous preparation.

4. The process as claimed in claim 2,
wherein reaction temperature is 20° C. to 80° C.

5. The process as claimed in claim 2,
wherein reaction time is between one hour and a few days.

6. The process as claimed in claim 2,
wherein the complexation occurs under atmospheric pressure and takes place under a protective-gas atmosphere and with the exclusion of daylight.

7. A cosmetic formulation comprising
a complex selected from the group consisting of β-cyclodextrin and α-tocopherol in a molar ratio of 2:1, and γ-cyclodextrin and α-tocopherol in a molar ratio of 2:1, and
an inert nontoxic carrier.

8.
A sunscreen cream consisting essentially of

| | |
|---|---|
| octyl palmitate | 2.50% by weight |
| octyl stearate | 3.50% by weight |
| polyglycerol-2 sesquiisostearate | 2.00% by weight |
| cyclomethicones, dimethiconol | 3.00% by weight |
| lauryldimethicone | 2.00% by weight |
| octyldimethicone ethoxyglycoside, | 12.00% by weight |
| cyclomethicone | |
| titanium dioxide | 5.00% by weight |
| polymethylsilsesquioxane | 1.00% by weight |
| zinc oxide | 2.00% by weight |
| γ-cyclodextrin complex 2:1, 13% | 1.52% by weight |
| α-tocopherol (prepared in accordance | |
| with Example 2b) | |
| glycerol | 2.00% by weight |
| methyl paraben | 0.10% by weight |
| sodium chloride | 0.40% by weight |
| water | 63.10% by weight; |
| | and |

wherein each percent by weight is based upon the total
weight of the sunscreen cream.

9.
An aftersun lotion consisting essentially of

| | |
|---|---|
| cetyl alcohol | 1.50% by weight |
| mineral oil | 5.00% by weight |
| stearic acid | 5.00% by weight |
| allantoin | 0.50% by weight |
| propylene glycol | 3.00% by weight |
| water | 67.05% by weight |
| cyclomethicones, dimethicones | 15.00% by weight |
| phenyltrimethicones | 2.00% by weight |
| γ-cyclodextrin complex 2:1, 13% | 0.38% by weight |
| α-tocopherol (prepared: Example 2b) | |
| phenoxyethanol, | 0.30% by weight |
| methyl paraben, butyl paraben, | |
| ethyl paraben, propyl paraben | |
| perfume | 0.30% by weight; |
| | and |

wherein each percent by weight is based upon the total
weight of the aftersun lotion.

10.
A liquid make-up consisting essentially of

| | |
|---|---|
| white beeswax | 2.70% by weight |
| polyglyceryl-2 sesquiisostearate | 2.40% by weight |
| dimethicone | 12.50% by weight |
| octyldimethicone ethoxyglucoside, | 11.00% by weight |
| cyclomethicone | |
| trimethylsiloxysilicate | 1.50% by weight |
| iron oxide | 1.46% by weight |
| talc | 5.00% by weight |
| titanium dioxide | 7.00% by weight |
| sodium chloride | 2.00% by weight |
| water | 53.39% by weight |
| methylchloroisothazolinone | 0.05% by weight |
| perfume | 0.30% by weight |
| γ-cyclodextrin complex 2:1, 13% | |
| α-tocopherol (prepared: Example 2b)) | 0.77% by weight; |
| | and |

wherein each percent by weight is based upon the total
weight of the liquid make-up.

11.
A body emulsion consisting essentially of

| | |
|---|---|
| glycerol monomyristate | 1.4% by weight |
| stearic acid | 1.2% by weight |
| cetyl alcohol | 0.5% by weight |
| isopropyl palmitate | 5% by weight |
| γ-cyclodextrin complex 2:1, 113% | 0.38% by weight |
| α-tocopherol (Example 2b) | |
| water distilled | 90.55% by weight |
| methyl paraben | 1% by weight; and |

wherein each percent by weight is based upon the total
weight of the body emulsion.
